(19) United States
(12) Patent Application Publication
(10) Pub. No.: US 2025/0318908 A1
(43) Pub. Date: Oct. 16, 2025

(54) REBONDING TRAY FOR ORTHODONTIC TREATMENT

(71) Applicant: LightForce Orthodontics, Inc., Burlington, MA (US)

(72) Inventor: Oisín Duggan, Somerville, MA (US)

(73) Assignee: LightForce Orthodontics, Inc., Burlington, MA (US)

(21) Appl. No.: 19/174,504

(22) Filed: Apr. 9, 2025

Related U.S. Application Data
(60) Provisional application No. 63/632,186, filed on Apr. 10, 2024.

(51) Int. Cl. A61C 7/14; A61C 7/00; G16H 10/60; G16H 20/40

(52) U.S. Cl. CPC A61C 7/146; A61C 7/002; G16H 10/60; G16H 20/40

(57) ABSTRACT

A rebonding tray being manufactured according to a method of manufacturing a rebonding tray, the method comprising: obtaining a first treatment configuration and a second treatment configuration, the first and second treatment configuration comprising positions of the target tooth and a neighboring tooth of the target tooth; determining a mapping between the first treatment configuration and the second treatment configuration, wherein the mapping comprises a path of tooth movement for the neighboring tooth of the target tooth and corrects for movement of the target tooth; determining an exclusion volume based on the mapping between the first treatment configuration and the second treatment configuration; determining a shape of a rebonding tray based on a shape of the target tooth and the exclusion volume; and manufacturing a rebonding tray based on the determined shape of the rebonding tray.

### CROSS-REFERENCE TO RELATED APPLICATIONS

This application claims the benefit of priority under 35 U.S.C. § 119(e) to U.S. Provisional Patent Application Ser. No. 63/632,186, filed on Apr. 10, 2024, and titled “REBONDING TRAY FOR ORTHODONTIC TREATMENT,” which is incorporated by reference herein in its entirety.

### BACKGROUND

Rebond trays are trays are orthodontic tools for supporting orthodontic brackets during the attachment of the bracket to a patient's tooth. Accordingly, rebond trays are used in connection with an orthodontic treatment plan to aid in the proper placement of brackets to a patient's tooth. When placed over a tooth, rebond trays help secure the bracket in place so that the bonding agent can set, bonding the bracket to the tooth.

### SUMMARY

The following is a non-limiting summary of some embodiments of the present application. Some embodiments provide for a method of designing a rebonding tray, the method comprising: using at least one computer hardware processor to perform: obtaining a first treatment configuration comprising a first position of a target tooth and a first position of a neighboring tooth of the target tooth; obtaining a second treatment configuration comprising a second position of the target tooth and a second position of the neighboring tooth of the target tooth; determining a mapping between the first treatment configuration and the second treatment configuration, wherein the mapping comprises a path of tooth movement for the neighboring tooth of the target tooth and corrects for movement of the target tooth; determining an exclusion volume based on the mapping between the first treatment configuration and the second treatment configuration; and determining a shape of the rebonding tray based on a shape of the target tooth and the exclusion volume.

Some embodiments provide for a system comprising at least one hardware processor; and at least one non-transitory computer-readable storage medium storing processor executable instructions that, when executed by the at least one computer hardware processor, cause the at least one computer hardware processor to perform a method for manufacturing a rebonding tray, the method comprising: obtaining a first treatment configuration comprising a first position of a target tooth and first position of a neighboring tooth of the target tooth; obtaining a second treatment configuration comprising a second position of the target tooth and a second position of the neighboring tooth of the target tooth; determining a mapping between the first treatment configuration and the second treatment configuration, wherein the mapping comprises the path of tooth movement for the neighboring tooth of the target tooth; determining an exclusion volume based on the mapping between the first treatment configuration and the second treatment configuration; and determining a shape of a rebonding tray based on a shape of the target tooth and the exclusion volume.

Some embodiments provide for at least one non-transitory computer-readable storage medium storing processor executable instructions that, when executed by at least one computer hardware processor, cause the at least one computer hardware processor to perform a method for manufacturing a rebonding tray, the method comprising: obtaining a first treatment configuration comprising a first position of a target tooth and first position of a neighboring tooth of the target tooth; obtaining a second treatment configuration comprising a second position of the target tooth and a second position of the neighboring tooth of the target tooth; determining a mapping between the first treatment configuration and the second treatment configuration, wherein the mapping comprises the path of tooth movement for the neighboring tooth of the target tooth; determining an exclusion volume based on the mapping between the first treatment configuration and the second treatment configuration; and determining a shape of a rebonding tray based on a shape of the target tooth and the exclusion volume.

Some embodiments provide for a rebonding tray, the rebonding tray being manufactured according to a method of manufacturing a rebonding tray, the method comprising: obtaining a first treatment configuration comprising a first position of a target tooth and first positions of a neighboring tooth of the target tooth; obtaining a second treatment configuration comprising a second position of the target tooth and second the neighboring tooth of the target tooth; determining a mapping between the first treatment configuration and the second treatment configuration, wherein the mapping comprises a path of tooth movement for the neighboring tooth of the target tooth and corrects for movement of the target tooth; determining an exclusion volume based on the mapping between the first treatment configuration and the second treatment configuration; determining a shape of a rebonding tray based on a shape of the target tooth and the exclusion volume; and manufacturing a rebonding tray based on the determined shape of the rebonding tray.

In some embodiments, the method further comprises: obtaining one or more additional treatment configurations between the first treatment configuration and the second treatment configuration, wherein each of the one or more additional treatment configurations comprise respective positions of the target tooth and a respective position of the neighboring tooth of the target teeth; and determining the exclusion volume comprises mapping between the first treatment configuration, the one or more additional treatment configurations, and the second treatment configuration.

In some embodiments, the first treatment configuration is determined based on a scan of the teeth in a patient's mouth.

In some embodiments, the neighboring tooth of the target tooth comprises one of the patient's other teeth located in the patient's jaw with the target tooth.

In some embodiments, the second treatment configuration comprises the target tooth positions for a patient's teeth at the end of an orthodontic treatment plan.

In some embodiments, the mapping between the first treatment configuration and the second treatment configuration comprises determining a transformation between the second treatment configuration and the first treatment configuration such that the second position of the target tooth, in the second treatment configuration, is aligned with the first position of the target tooth, in the first treatment configuration.

In some embodiments, the transformation between the second treatment configuration and the first treatment configuration is determined such that a bracket position of the target tooth in the second position is aligned with a bracket position in the first position.

In some embodiments, the transformation between the second treatment configuration and the first treatment configuration is determined such that the centroid position of the target tooth in the second position is aligned with the centroid of the target tooth in the first position.

In some embodiments, determining the exclusion volume comprises combining the volume of the neighboring tooth of the target tooth with a gingiva volume.

In some embodiments, the gingiva volume is determined based on the scan of the teeth in a patient's mouth and a transformation.

In some embodiments, the method further comprises manufacturing a rebonding tray based on the determined shape of the rebonding tray, wherein manufacturing the rebonding tray comprises using additive manufacturing to manufacture the rebonding tray.

In some embodiments, the exclusion volume includes a first position and a second position of hardware mounted to the target tooth and/or the neighboring tooth of the target tooth in the first treatment configuration and the second treatment configuration.

In some embodiments, determining the exclusion volume based on the mapping between the first treatment configuration and the second treatment configuration further comprises using one or more additional teeth located in the patient's jaw with the target tooth.

### BRIEF DESCRIPTION OF THE DRAWINGS

FIG. 1A illustrates an example of a rebonding tray configuration 100, in accordance with some embodiments of the technology described herein.

FIG. 1B illustrates a side view of rebonding tray 102, in accordance with some embodiments of the technology described herein.

FIG. 1C is a top view of rebonding tray 102, in accordance with some embodiments of the technology described herein.

FIG. 2 illustrates an example of rebonding tray 200, in accordance with some embodiments of the technology described herein.

FIG. 3 illustrates a flowchart of process 300 for designing a rebonding tray, in accordance with some embodiments of the technology described herein.

FIG. 4 illustrates an example of the result of a mapping between the first treatment configuration and the second treatment configuration, in accordance with some embodiments of the technology described herein.

FIG. 5A illustrates an example of a first treatment configuration 500, in accordance with some embodiments of the technology described herein.

FIG. 5B illustrates an example of the motion of the neighboring teeth shown in FIG. 5A, in accordance with some embodiments of the technology described herein.

FIG. 5C illustrates an example exclusion volume configuration 500, in accordance with some embodiments of the technology described herein.

FIG. 6A illustrates an example of rebonding tray 600, in accordance with some embodiments of the technology described herein.

FIG. 6B illustrates the example rebonding tray 600 of FIG. 6A placed over a target tooth.

FIG. 7 illustrates an example implementation of a computer system 700 that may be used in connection with any of the embodiments of the disclosure provided herein.

### DETAILED DESCRIPTION

The inventors have developed technologies for improving the manufacture of rebonding trays for use in orthodontic treatments. The improved technologies include systems and methods for manufacturing rebonding trays, as well as the resulting manufactured tray, for improving rebonding of orthodontic brackets during the progression of an orthodontic treatment.

Orthodontic procedures use tools such as orthodontic hardware, including brackets and archwires to apply forces to teeth to cause their movement towards desired treatment positions. During a procedure, brackets may be attached directly to a patient's teeth and an archwire attached to the brackets. In some cases, the arch wire may have a predefined shape that represents a desired final configuration of the patient's teeth (e.g., an arrangement of the patient's teeth relative to one another in the patient's mouth). When the archwire is attached to brackets on the patient's teeth in their initial configuration, it may be stretched or distorted from that predefined shape in order to connect to each of the brackets. Once distorted, elastic forces applied by the archwire pull the wire towards its predefined shape thereby applying forces to the brackets and teeth, and gradually bring the teeth into the desired arrangement.

Indirect bonding (IBD) trays are commonly used at the beginning of treatment to aid in bonding the brackets to the patient's teeth. IBD trays used at the beginning of treatment are generally segmented such that the tray can bond to multiple teeth at once, ensuring appropriate positioning of the brackets along the initial patient archline. Over the course of treatment, brackets may become detached from the patient's tooth due to strain applied to the bracket (e.g., strain applied during eating). If left untreated for too long, detachments can result in treatment delays, which can extend the time required for a patient to complete an orthodontic treatment plan. Accordingly, to facilitate rebonding of the bracket to the patient's tooth, rebonding trays can be included with the indirect bonding tray or subsequently provided for use with an orthodontic treatment plan.

The inventors have appreciated that by providing the rebonding trays along with an indirect bonding tray that is used for initial bonding or separately in advance of a detachment, the time for rebonding can be minimized because, at the time of detachment, the reattachment can be performed without delay since it does not require the separate manufacturing and/or shipping of rebonding trays. However, while the indirect bonding tray used at the start of treatment may be configured to position brackets for all of the patient's teeth, or some subset thereof, a rebonding tray generally cannot reposition multiple brackets at the same time. Further, the indirect bonding tray used at the beginning of treatment is designed based on the initial position of the teeth in the patient's mouth. However, as the brackets may become detached at any point during treatment, it is not possible to know what the positional relationship between the teeth and/or orthodontic appliances will be at the time of detachment. By extension, it is difficult to design a rebonding tray which is configured to facilitate bonding to multiple teeth at the same time, because the tray would need to accommodate the positional relationship between the teeth be able to be properly positioned over each tooth for bonding. Accordingly, rebonding trays may be designed for specific teeth to attach a single bracket to the target tooth to which the bracket had detached.

The inventors have recognized and appreciated that a single tooth bonding tray, relative to a multi-tooth bonding tray, has fewer support points to enforce its positioning resulting in less stable positioning of the bracket to the tooth. Accordingly, the use additional support material, to increase the structural stability of the rebond tray, may reduce distortions of the shape of the rebonding tray and may, by extension, facilitate accurate attachment of the bracket to a tooth. However, increasing the amount of support material being used also increases the volume of the rebonding tray. The increased volume of the rebonding tray may prohibit the use of the rebonding when the volume around the target tooth, occupied by the rebonding tray during use, is obscured by the neighboring teeth and/or neighboring orthodontic appliances. One solution to the lack of clearance between the rebonding tray and the neighboring teeth is to adjust the tray by cutting away portions of the rebonding tray to provide clearance for the neighboring teeth. However, the process of cutting away portion of the rebonding tray weakens the structural integrity of the rebonding tray and runs the risk of deforming the tray. By extension, cutting away portions of the tray make reliable positioning of the rebonding tray on the tooth more difficult. As another potential solution, rebonding trays may be produced, as needed, in response to a bracket detaching at a particular stage of treatment. However, the process of producing rebonding trays as needed generally requires taking note of the current teeth positions, which may require additional scanning of a patient's mouth. Additionally, time is spent to manufacture the new rebonding tray, which may result in additional treatment delays.

Thus, to improve rebonding, the inventors have developed rebonding trays that may be used over the progression of an orthodontic treatment by including recesses in the walls of the rebonding tray based on the orthodontic treatment plan such that the trays do not require adjustments to the shape of the tray during treatment. The recesses may be based on the positions of the neighboring teeth (and any associated orthodontic appliances, such as brackets, tubes, etc.) through treatment, such that the clearance between the rebonding tray and the neighboring tooth is maintained when the teeth move relative to one another. For a particular tooth, a rebonding tray is designed to account for the movement of the neighboring teeth and bracket movement. To account for the movement of the neighboring teeth and brackets, the movement during an orthodontic treatment is modeled between a first treatment position and a second treatment position such that an exclusion space representing the positions of the neighboring teeth and brackets may be determined and accounted for in the rebonding tray design. For example, by removing rebonding tray material which would intersect with the exclusion space, the rebonding tray may be used with the target tooth through treatment.

Accordingly, some embodiments provide for a method of designing a rebonding tray, the method comprising: using at least one computer hardware processor to perform: obtaining a first treatment configuration comprising a first position of a target tooth and a first position of a neighboring tooth of the target tooth (e.g., first positions being determined based on a scan of a patient's mouth); obtaining a second treatment configuration comprising a second position of the target tooth and a second position of the neighboring tooth of the target tooth (e.g., second positions being the target tooth positions for a patient's teeth at a later stage of the orthodontic treatment plan); determining a mapping between the first treatment configuration and the second treatment configuration, wherein the mapping comprises a path of tooth movement for the neighboring tooth of the target tooth and corrects for movement of the target tooth (e.g., determining a transformation between the second treatment configuration and the first treatment configuration such that the second position of the target tooth, in the second treatment configuration, is aligned with the first position of the target tooth, in the first treatment configuration); determining an exclusion volume based on the mapping between the first treatment configuration and the second treatment configuration; and determining a shape of a rebonding tray based on a shape of the target tooth and the exclusion volume.

In some embodiments, the method further comprises: obtaining one or more additional treatment configurations between the first treatment configuration and the second treatment configuration, wherein each of the one or more additional treatment configurations comprise respective positions of the target tooth and a respective position of the neighboring tooth of the target teeth; and determining the exclusion volume comprises mapping between the first treatment configuration, the one or more additional treatment configurations, and the second treatment configuration.

In some embodiments, the neighboring tooth of the target tooth comprises one of the patient's other teeth located in the patient's jaw with the target tooth.

In some embodiments, the transformation between the second treatment configuration and the first treatment configuration is determined such that a bracket position of the target tooth in the second position is aligned with a bracket position of the target tooth in the first position.

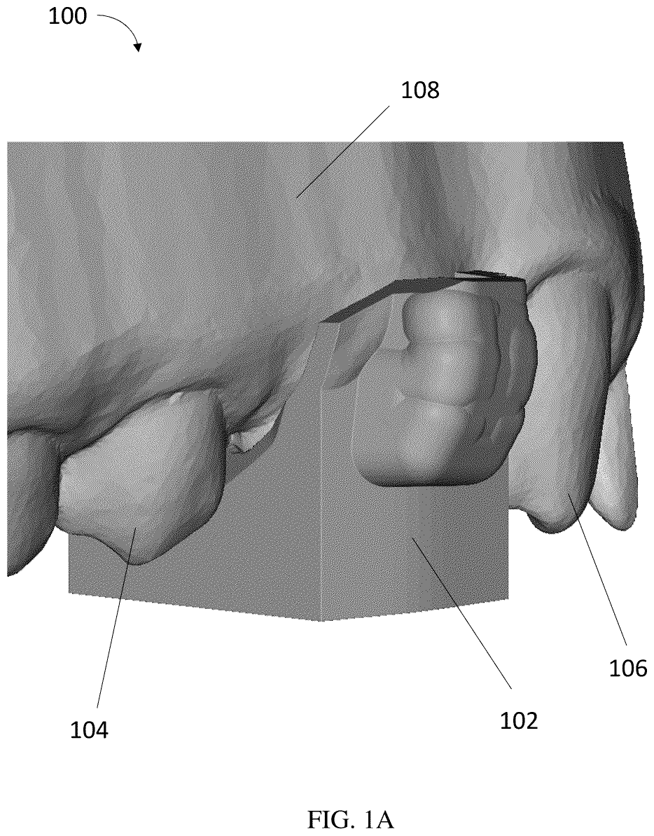

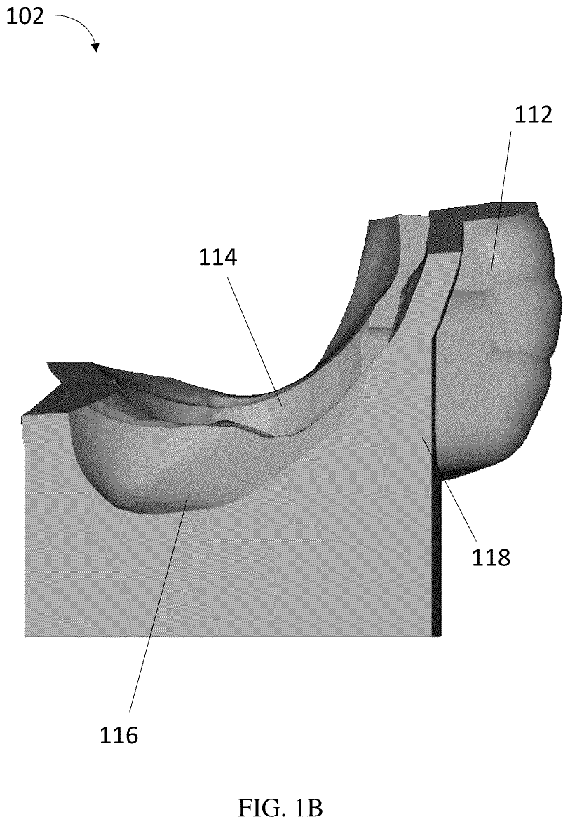

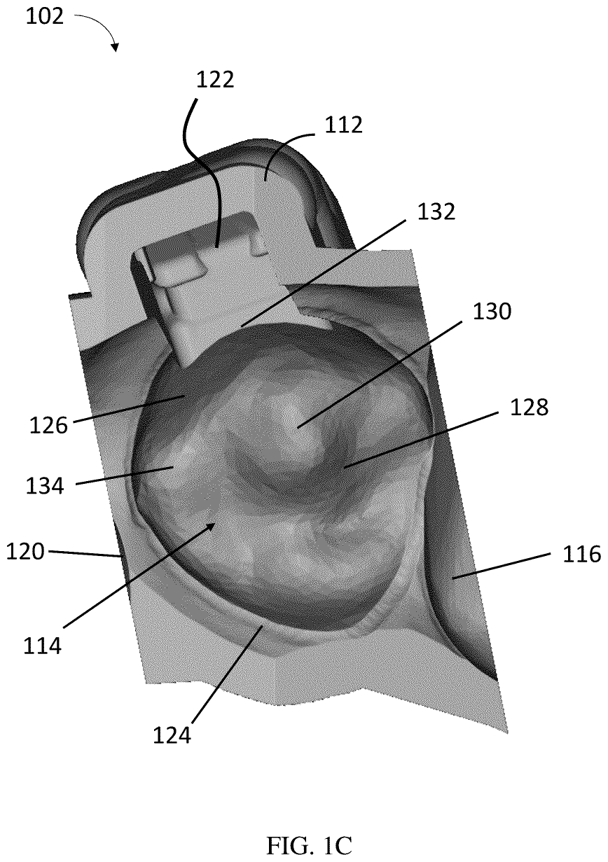

FIG. 1A illustrates an example of a rebonding tray configuration 100, in accordance with some embodiments of the technology described herein. Rebonding tray configuration 100 includes rebonding tray 102 and patient's jaw 108. The patient's jaw includes a target tooth covered and neighboring teeth 104 and 106. The target tooth is covered by rebonding tray 102. Rebonding tray 102 is in placed over the target tooth such that the rebonding tray is in a bonding configuration. The bonding configuration may be used for attaching a bracket at an intended bracket position on the target tooth, while the target tooth is positioned between neighboring teeth 104 and 106.

As shown in FIG. 1A, rebonding tray 102 is positioned over the target tooth. To facilitate the positioning of the rebonding tray over the target tooth, an internal cavity is configured to receive the target tooth within the rebonding tray. The internal cavity includes protrusions and recesses such that the rebond tray conforms to surface features of the tooth when properly positioned. Through contact with the surface features of the tooth, the movement of the rebonding tray is restricted such that a bracket supported by the rebonding tray is held in place. For example, through contact with the surface features of the target tooth, the rebond tray is restricted from rotational movement. Accordingly, when a bracket is included in the bracket cavity for bonding with the target tooth, the bracket is held in place against the surface of the tooth with its lateral movement restricted. Additionally, a finite depth of the internal cavity of the rebond tray may restrict the vertical movement of the rebond tray and, by extension, a bracket located within the bracket cavity. For example, based on the depth of the internal cavity, the rebond tray will be set against the crown of the tooth, limiting the vertical movement of the rebonding tray. When pressed against the crown of the tooth, the rebonding tray will position the bracket at the appropriate height on the tooth.

FIG. 1B illustrates a side view of rebonding tray 102, in accordance with some embodiments of the technology described herein. Rebonding tray 102 includes bracket cavity 112, internal cavity 114, side wall 118, and recess 116. Bracket cavity 112 is configured to retain a bracket for mounting the bracket to a target tooth. Bracket cavity 112 is positioned, relative to the front surface of the tooth, such that the bracket cavity supports the bracket in position for bonding during the bonding process. In some embodiments, the bracket cavity 112 protrudes from front wall 118 of the rebonding tray. The protrusion of bracket cavity 112 provides a volume for accommodating the bracket.

Internal cavity 114 is configured to conform to surface features of the target tooth, in accordance with some embodiments of the technology described herein. By conforming the internal surface of internal cavity 114 to surface features of the target tooth, internal cavity 114 ensures reliable positioning of the rebonding tray—and by extension, a bracket for mounting to the tooth—over the surface of the tooth.

Recess 116 is configured to accommodate the position of a neighboring tooth, in accordance with some embodiments of the technology described herein. Recess 116 provides clearance between the outer surface of rebonding tray 102 and a neighboring tooth of the target tooth. In some embodiments, recess 116 is shaped to provide clearance between the outer surface of rebonding tray 102 and a neighboring tooth throughout an entire orthodontic treatment. For example, recess 116 is configured to provide clearance between the rebonding tray and a neighboring tooth at the beginning of treatment by providing space to accommodate the volume of the neighboring tooth in its initial position at the start of treatment. Furthermore, the rebonding tray is further configured to provide clearance with the neighboring tooth at the end of treatment by providing space to accommodate the volume of the neighboring tooth in its final position at the end of treatment. Similarly, the rebonding tray is configured to provide clearance for the neighboring tooth at additional treatment stages between the beginning and end of treatment.

FIG. 1C is a top view of rebonding tray 102, in accordance with some embodiments of the technology described herein. As shown in FIG. 1C, the top view of rebonding tray 102 illustrates the shape and structure of internal cavity 114. Internal cavity 114 includes protrusions 130 and 134 for conforming to the grooves on the surface of the target tooth. Additionally, internal cavity 114 includes grooves 126 and 128 for conforming to the cusps on the surface of the target tooth.

In some embodiments, rebond tray 102 includes a beveled edge 124 around the edge of internal cavity 114. The beveled edge may be configured with a curvature. In some embodiments, the beveled edge 124 is shaped to provide clearance between the rebond tray and the gingiva around the target tooth.

Rebond tray 102 includes bracket cavity 112, in accordance with some embodiments of the technology described herein. Bracket cavity 112 includes groove 122 for retaining a bracket in bracket cavity 112 while the bracket is bonded to the target tooth. The bracket cavity 122 retains a bracket such that a bonding surface of the bracket makes contact, for bonding with the surface of the target tooth, at interface 132.

As shown in FIG. 1C, rebonding tray 102 includes a first recess 116 to accommodate the position of a first neighboring tooth and recess 120 to accommodate the position of a second neighboring tooth. Recesses 116 and 120 are designed to enable use of rebonding tray 102 throughout the duration of an orthodontic treatment while retaining sufficient support material for reliable positioning of a bracket on the surface of a target tooth. First recess 116 is configured as described above in connection with FIG. 1B.

Second recess 120 is configured to accommodate the position of a second neighboring tooth, in accordance with some embodiments of the technology described herein. Recess 120 provides clearance between the outer surface of rebonding tray 102 and a second neighboring tooth of the target tooth. The second neighboring tooth being located on an opposite side of the target tooth relative to the target tooth accommodated by recess 116. In some embodiments, recess 120 is configured similarly to recess 116, such that recess 120 is configured to provide clearance between the outer surface of rebonding tray 102 and the second neighboring tooth throughout an entire orthodontic treatment. However, as the initial and final positions of the second neighboring tooth are different than the first neighboring tooth, recess 120 is shaped differently from recess 116.

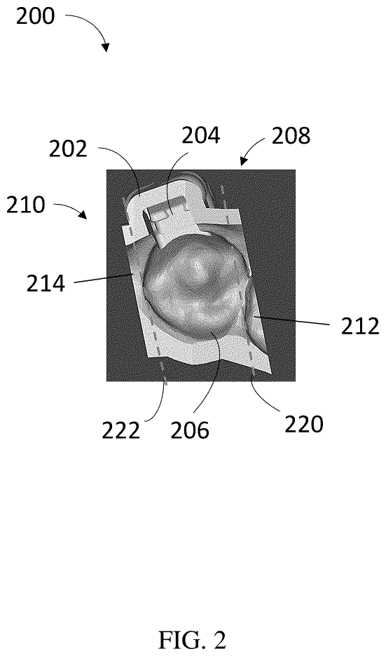

FIG. 2 illustrates an example of rebonding tray 200, in accordance with some embodiments of the technology described herein. Rebonding tray 200 includes bracket cavity 202, bracket retention groove 204, internal cavity 206, first side portion 208 and second side portion 210. The first side portion 208 includes recess 212 to accommodate a first neighboring tooth, when the rebonding tray is placed over a target tooth. Similarly, the second side portion 210 includes rounded edge 214 to accommodate a second neighboring tooth, when the rebonding tray is placed over a target tooth.

Absent the accommodating features 212 and 214, the rebonding tray 202 would not fit over the target tooth without being obstructed by the neighboring teeth. Accordingly, the rebonding tray would need to be trimmed or sanded down to remove the material that would be obscured by the neighboring teeth. Using conventional methods, the side regions 208 and 210 would be removed, for example by using a scalpel and slicing them off, between the edge of the rebonding tray and dashed lines 220 and 222. The removal of side regions 208 and 210 decreases the thickness of the side walls and, by extension, weakens the structural integrity of the rebonding tray. Depending on the amount of side region removed, the removal may reduce the number of points of contact between the rebonding tray and the target tooth. Accordingly, the removal of tray material may result in the rebonding tray being more deformable, thus decreasing the accuracy of bracket positioning. Additionally, the reduced points of contact between the rebonding tray and the target tooth may result in less reliable positioning of the rebonding tray on the target tooth, also decreasing the accuracy of bracket positioning. Accordingly, the incorporation of the accommodating features provides for additional support material to be included with the rebonding tray. Additionally, the design of the accommodating features to provide clearance for neighboring teeth throughout the course of treatment enables a single rebonding tray to be used for the target tooth, improving treatment efficiencies, as described herein.

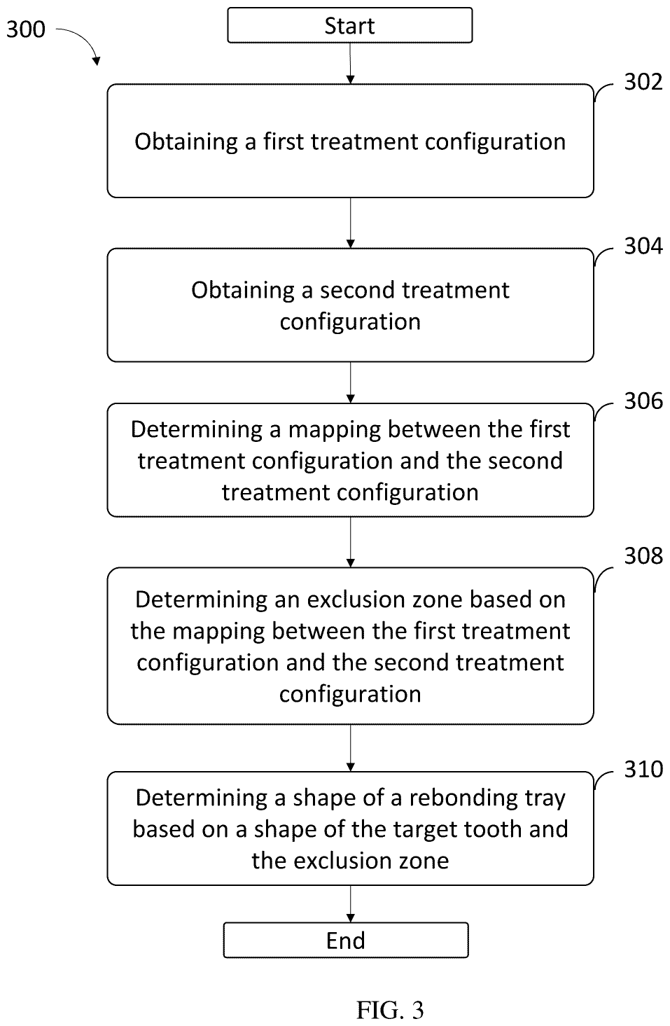

FIG. 3 illustrates a flowchart of process 300 for designing a rebonding tray, in accordance with some embodiments of the technology described herein. Prior to the start of process 300, a digital model may be acquired of the patient's mouth. Any suitable modeling technique may be used as aspects of the technology described herein are not limited in this respect. For example, any modeling method which can capture the shape of the teeth and the spatial positioning of the teeth relative to one another may be used. As non-limiting examples, X-ray imaging, optical imaging, dental impressions which are then digitized, or some combination thereof may be used to obtain/generate the scan of the patient's mouth.

Additionally, prior to the start of process 300, an orthodontic treatment plan may be generated. The orthodontic treatment plan may include the planned movement of the patient's teeth between their initial positions and desired final positions. The initial positions may be obtained from the digital model of the patient's mouth. The final positions may be obtained from a desired arch shape. The desired arch shape may be provided by an orthodontist or by a user of an orthodontic treatment planning software platform. Alternatively, the desired arch shape may be determined by the orthodontic treatment planning software.

Process 300 starts at act 302 by obtaining a first treatment configuration, in accordance with some embodiments of the technology described herein. The first treatment configuration includes a first position of a target tooth and a first position of a neighboring tooth of the target tooth. The first position of the target tooth and the first position of the neighboring tooth of the target tooth may be obtained from a digital model of the patient's mouth. In some embodiments, the positions of all of the patient's teeth may be obtained.

In some embodiments, the neighboring tooth of the target tooth includes one of the patient's other teeth, located in the patient's jaw with the target tooth. For example, if the target tooth is in the patient's upper jaw, then the neighboring teeth may include one of the other teeth located in the patient's upper jaw. In some embodiments, the neighboring teeth may include the immediate neighbor of the target tooth, e.g., one or both of the closest teeth on either side of the target tooth. In some embodiments, the neighboring teeth may include the first two closest teeth on either side of the target tooth.

In some embodiments, the first treatment configuration is determined based on a scan of the patient's teeth. For example, based on a scan of the patient's teeth, the locations of teeth within the scan may be identified by a user of an orthodontic treatment planning software platform by identifying a region of the scan which are associated with a particular tooth. As another example, the locations of teeth within the scan may be identified by the orthodontic treatment planning software platform automatically using image identification algorithms. Image identification algorithms may include trained machine learning models configured to identify teeth from a scan of a patient's teeth.

The positions of the patient's teeth may be obtained in any suitable format. For example, a 3D model file such as a standard tessellation language (STL), polygon file format (PLY), or object file format (OBJ) file type. Other file types may be used as aspects of the technology described herein are not limited in this respect.

Next, process 300 proceeds to act 304 by obtaining a second treatment configuration, in accordance with some embodiments of the technology described herein. The second treatment configuration including a second position of the target tooth and a second position of the neighboring tooth of the target tooth. The second position of the target tooth and the second position of the neighboring tooth of the target tooth may be obtained from a treatment plan of the patient's teeth. In some embodiments, obtaining as second treatment configuration includes receiving a digital model of the patient's teeth generated in accordance with the treatment plan for the patient's teeth. In some embodiments,

In some embodiments, act 304 may further include obtaining one or more additional treatment configurations between the first treatment configuration and the second treatment configuration. The one or more additional treatment configurations include respective positions of the target tooth and respective positions of the neighboring tooth of the target tooth.

Next process 300 proceeds to act 306 by determining a mapping between the first treatment configuration and the second treatment configuration, in accordance with some embodiments of the technology described herein. Determining a mapping between the first treatment configuration and second treatment configuration includes determining a transformation between the second treatment configuration and the first treatment configuration such that the second position of the target tooth, in the second treatment configuration, is aligned with the first position of the target tooth, in the first treatment configuration.

In some embodiments, the transformation between the second treatment configuration and the first treatment configuration is a transformation to align the bracket position from the second treatment configuration to the bracket position on the first treatment configuration. In some embodiments, the transformation between the second treatment configuration and the first treatment configuration is a transformation to align one or more feature points of the tooth in the second treatment configuration to the position of the corresponding feature points of the tooth in the first treatment configuration. For example, the transformation between the second treatment configuration and the first treatment configuration is a transformation to align the centroid position of the target tooth in the second position with the centroid of the tooth in the first configuration.

The determined mapping is applied to all of the teeth in the treatment configuration such that the arrangement spatial between teeth in the treatment configuration is maintained when the mapping is applied. Accordingly, the movement of the target tooth is compensated for by the alignment of the target tooth in the second treatment configuration to the alignment of the target tooth in the first treatment configuration. By applying the same mapping to the other teeth in the second treatment configuration, the movement of teeth relative to the position of the target tooth is preserved.

Next process 300 proceeds to act 308 by determining an exclusion zone based on the mapping between the first treatment configuration and the second treatment configuration, in accordance with some embodiments of the technology described herein. The exclusion zone may be determined by the positions of teeth along the mapping between the first and second treatment configuration.

In some embodiments, the exclusion zone is an exclusion volume determined by combining the volumes of the respective teeth. Accordingly, the exclusion volume may include the volumes occupied by a neighboring tooth, of the target tooth, in the first treatment configuration and the second treatment configuration. For example, using the FDI notation and taking tooth 18 as the target tooth, the volume of the neighboring tooth, tooth 17, may be used to determine the exclusion volume. To determine the exclusion volume, the first volume occupied by tooth 17 in the first treatment configuration is combined with the second volume occupied by the tooth 17 in the second treatment configuration. In some embodiments, the exclusion volume may include the volumes occupied by two neighboring teeth. For example, using the FDI notation and taking tooth 14 as the target tooth, the volume of the neighboring teeth, tooth 13 and tooth 15, may be used to determine the exclusion volume. To determine the exclusion volume the first volumes occupied by tooth 13 and tooth 15 in the first treatment configuration are combined with the second volumes occupied by tooth 13 and tooth 15 in the second treatment configuration.

In some embodiments, the exclusion volume is determined by combining the 3D surface of the neighboring tooth in the first treatment configuration with the 3D surface of the neighboring tooth in the second treatment configuration. The 3D surfaces may be combined using any suitable technique for merging 3D representations. For example, a union function may be used to merge the surfaces of the teeth in the first treatment configuration with the surfaces of the teeth in the second treatment configuration.

In some embodiments, the exclusion volume is determined by determining a path of tooth movement for the neighboring tooth of the target tooth. In some embodiments, the path of tooth movement includes the first position of the neighboring tooth position and the final position of the neighboring tooth. In some embodiments, the path of tooth movement includes modeling a trajectory between the first tooth position of the neighboring tooth and the second position of the neighboring tooth. For example, the trajectory may be represented by a discretized set of positions determined from specific treatment configurations between an initial treatment configuration and a final treatment configuration.

As another example, the trajectory may be described by an analytical expression representing the trajectory of the neighboring tooth between the initial position and the final position of the neighboring tooth. The analytic expression may represent the change of position of a particular feature point of the tooth across the duration of the treatment between the initial position and the final position.

In some embodiments, the initial position and the final position represent a subset of the movements of the neighboring tooth included in the treatment plan. For example, the initial position may be a first position occurring earlier in treatment but not necessarily at the start of treatment. Additionally, the final position may be a second position occurring later in treatment but not necessarily at the end of treatment. In addition to the trajectory of the neighboring tooth, a volume centered on the trajectory is included in the trajectory representing the space occupied by the tooth. In some embodiments, the volume has the same shape as the tooth. In some embodiments, a generic shape may be used to approximate the space occupied by the tooth.

In some embodiments, the exclusion zone is a three-dimensional surface. The three-dimensional surface may represent the boundary of an exclusion volume, such as the exclusion volume described above. Alternatively, the three-dimensional surface may represent a closest surface based on the mapping between the first and second treatment configuration. For example, the three-dimensional surface may represent the closest surface of one or more neighboring teeth in the mapping.

In some embodiments, the exclusion zone is one or more exclusion planes. The exclusion planes may represent linear boundaries based on the mapping between the first treatment configuration and the second treatment configuration. For example, the linear boundaries may represent planes positioned between the target tooth and the closest point of one or more neighboring teeth in the mapping.

In some embodiments where one or more additional treatment configurations are obtained, determining the exclusion zone includes mapping the first treatment configuration, the one or more additional configurations, and the second treatment configuration. For example, each of surfaces from each treatment configuration may be joined using a union function for 3D surfaces.

In some embodiments, the exclusion zone includes the patient's gingiva. During the course of treatment, the patient's gingiva may become inflamed due to the movement of the teeth. Accordingly, the patient's gingiva may occupy a larger zone during treatment then it does in an initial scan of the patient's mouth. To account for the variable size of the patient's gingiva, the position of the gingiva may be offset by a factor to accommodate swelling. Additionally, or alternatively, to account for the variable size of the patient's gingiva, the surface corresponding the patient's gingiva may be transformed by an enlargement factor. In some embodiments, the transformation may include scaling the gingiva surface by the enlargement factor. In some embodiments, the transformation may include thickening the gingiva surface by the enlargement factor. For example, thickening the gingiva surface may include increase a thickness parameter associated with the 3D model of the gingiva. As another example, thickening the gingiva surface may include applying a uniform offset over the gingiva surface. In some embodiments, the transformation may include translation the gingiva surface by a distance represented by the enlargement factor. For example, the enlargement factor may represent a swelling of the gingiva corresponding to the gingiva line sitting higher on the tooth. The translation may move the gingiva surface such that it corresponds to the swollen gingiva line on the tooth. In some embodiments, the transformation may include rotation of the gingiva surface. In some embodiments, one or more combination of scaling, thickening, translating, and/or rotation may be used. In some embodiments, additional transformations may be used, as aspects of the technology described herein are not limited in this respect.

Next process 300 proceeds to act 310 by determining a shape of a rebonding tray based on a shape of the target tooth and the exclusion volume, in accordance with some embodiments of the technology described herein. The shape of the rebonding tray is determined based in part on the target tooth. The rebonding tray is shaped to conform to the surface of the target tooth. The spatial relationship of the cavities and protrusions on the rebonding tray is determined by the spatial relationship of the cusps and grooves of the target tooth, respectively. For example, the rebonding tray includes cavities positioned to receive cusps of the target tooth when the rebonding tray is placed over the target tooth. The depth of the cavity corresponds to the depth of the height of the cusp of the target tooth. Similarly, the rebonding tray include protrusions configured to fit within grooves of the target tooth when the rebonding tray is placed over the target tooth. The height of the protrusions corresponds to the depth of the groove of the target tooth. In some embodiments, the shape of the rebonding tray conforms to a sub-set of the surfaces of the target tooth. For example, the outer surface of the tooth and the top of the cusps of the tooth.

The shape of the rebonding tray is based in part on the exclusion volume, in accordance with some embodiments of the technology described herein. Portions of the rebonding tray which intersect with the exclusion volume are removed from the rebonding tray design. For example, as a first approximation of the rebonding tray, the design may start with a rectangular prism based on the size of maximum diameter of the tooth. The rectangular prism may be sized in accordance with the diameter of the tooth along two perpendicular axes such that the rectangular prism encompasses the outer surface of the tooth within its volume and a bracket to be bonded to the front surface of the tooth. The rectangular prism may include an additional buffer distance such that the walls of the rectangular prism have a minimum thickness that extends beyond the outer surface of the tooth and bracket. The internal volume of the rectangular prism may be removed based on the shape of the target tooth and bracket, such that the inner surface of the rebonding tray conforms to the outer surface of the target tooth with the bracket positioned at the desired bonding location on the surface of the target tooth, as described herein. Portions of the rectangular prism which intersect with the exclusion surface are removed from the rectangular prism. A subtract function may be used to subtract the volume encompassed by the volume of the exclusion surface from the model of the rectangular prism.

In some embodiments, other shapes may be used as the starting shape for the rebonding tray. For example, an ellipsoid may be used as the initial shape of the tooth. Other starting shapes may be used as aspects of the technology described herein are not limited in this respect.

In some embodiments, for the purposes of determining the shape of the rebonding tray, the bracket designed to be bonded to the target tooth may be considered as part of the volume of the tooth positioned at the bonding location on the surface of the tooth.

In some embodiments, an extra clearance factor may be added to the exclusion zone. For example, a 1%, 2%, 5%, or 10% enlargement of the exclusion surface may be applied prior to generating the shape of the rebonding tray. As another example, an offset of 0.1-5 mm may be used to provide for clearance of the exclusion zone with the rebonding bracket.

Following act 310, process 300 concludes. After the conclusion of process 300, the rebonding tray may be manufactured using additive manufacturing techniques, in accordance with some embodiments of the technology described herein. For example, the rebonding tray may be printed by an additive manufacturing device based on a model generated by process 300. Any suitable additive manufacturing device may be used, as aspects of the technology described herein are not limited in this respect. Additionally, manufacture of the rebonding tray is not limited to additive manufacturing techniques, as any suitable manufacturing technique may be used.

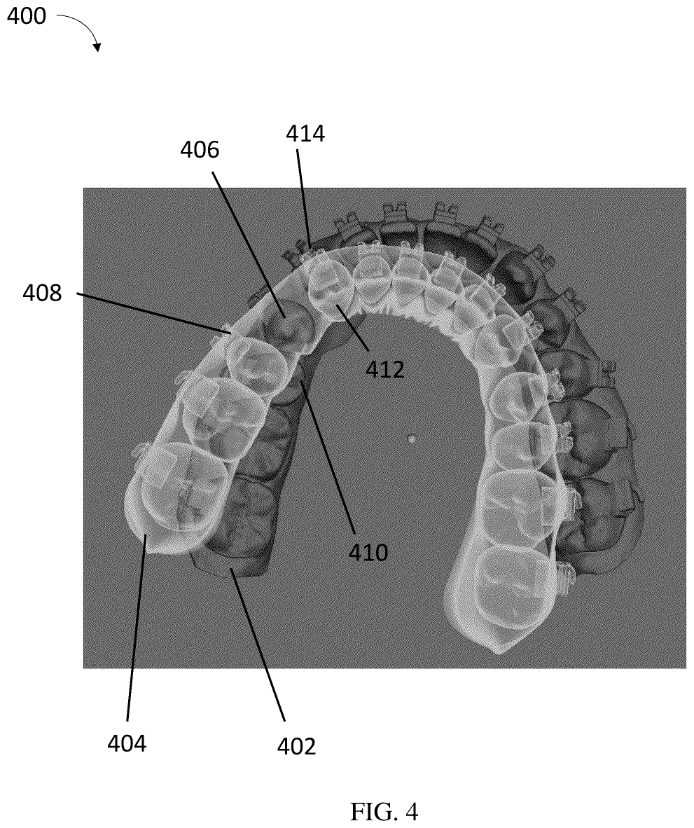

FIG. 4 illustrates an example of the result of a mapping between the first treatment configuration and the second treatment configuration, in accordance with some embodiments of the technology described herein. Mapping 400 includes first treatment configuration 402 and second treatment configuration 404. As shown in FIG. 4, the mapping is determined such that target tooth position 406 in the first treatment configuration 402 is mapped to the target tooth position in the second treatment configuration 404.

In some embodiments, first treatment configuration 402 includes first positions for all the patient's teeth in the lower jaw. Second treatment configuration 404 includes second positions for all the patient's teeth in the lower jaw. As shown in FIG. 4, each of the first treatment configuration 402 and second treatment configuration 404 include the patient's gingiva. In some embodiments, for computing the exclusion volume, first treatment configuration 402 includes neighboring teeth 410 and 414. Similarly, second treatment configuration 404 includes neighboring teeth 408 and 412. The exclusion volume used for determining the shape of a rebonding tray of target tooth 406 is based on the volumes occupied by the neighboring teeth in the first configuration 410 and 414 as well as the volumes occupied by the neighboring teeth in the second configuration 408 and 412. In some embodiments, the volumes 408 and 410 may be merged to form an exclusion volume on the left side of the target tooth. The volumes 414 and 412 may be merged to form an exclusion volume on the right side of the target tooth. In some embodiments, additional treatment configurations for the neighboring teeth may be included in determining the exclusion volume.

In some embodiments, the first treatment configuration and the second treatment configuration include the brackets for the corresponding teeth. Accordingly, the mapping between the first treatment configuration and the second treatment configuration may be based on an alignment of the position of the bracket for the target tooth in the second treatment configuration to the bracket position for the target tooth in the first treatment configuration.

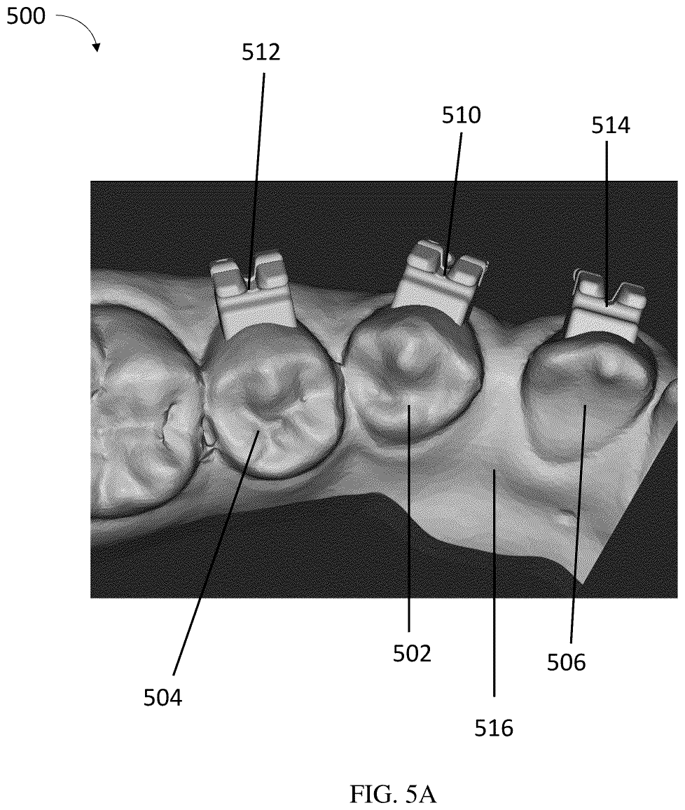

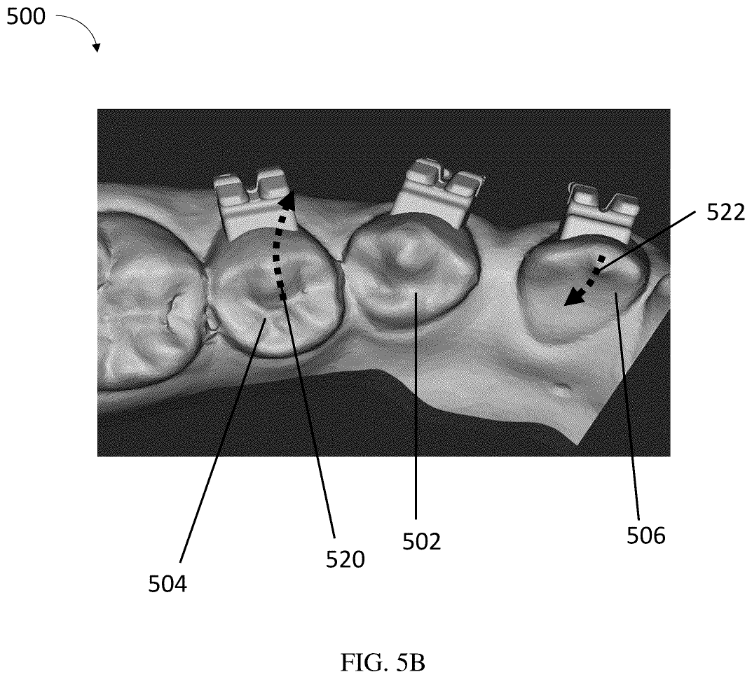

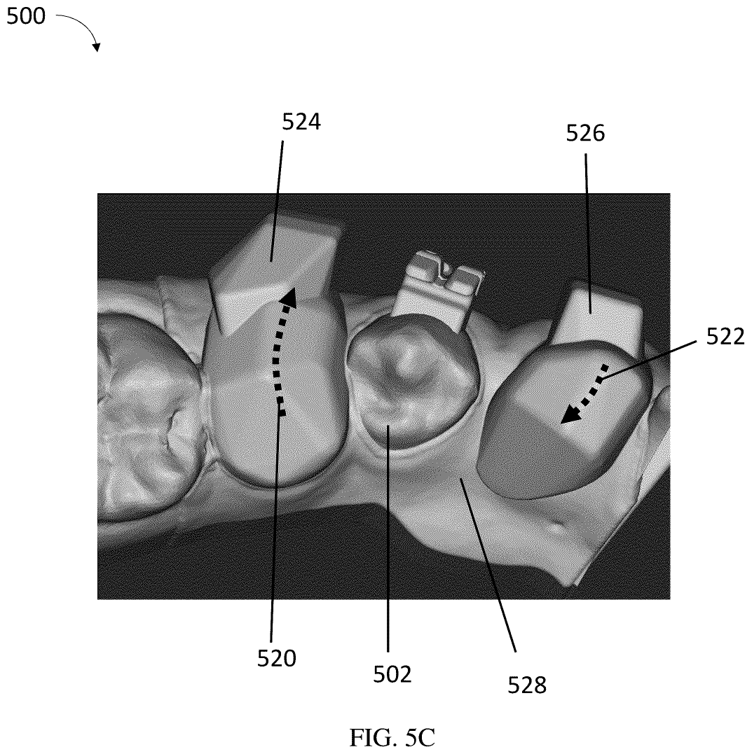

FIG. 5A illustrates an example of a first treatment configuration 500, in accordance with some embodiments of the technology described herein. First treatment configuration 500 includes target tooth 502 and neighboring teeth 504 and 506. The brackets associated with the target tooth and neighboring teeth are included in the first treatment configuration. Target tooth 502 includes bracket 510 positioned at the bonding position in accordance with an orthodontic treatment plan. Neighboring tooth 504 includes bracket 512 positioned at the bonding position in accordance with an orthodontic treatment plan. Neighboring tooth 506 includes bracket 514 positioned at the bonding position in accordance with an orthodontic treatment plan. In some embodiments, the first treatment configuration includes the patient's gingiva 516.

In the illustrated example of FIG. 5A, the target tooth is the LL4 tooth. Accordingly, the neighboring teeth include the LL3 tooth and the LL5 tooth. In other embodiments, only a single neighboring tooth may be included. For example, when the target tooth is the back tooth in the patient's jaw. As another example, when a neighboring tooth is missing, such as in the case of an extraction.

FIG. 5B illustrates an example of the motion of the neighboring teeth shown in FIG. 5A, in accordance with some embodiments of the technology described herein. As described in FIG. 5A, the target tooth 502 is shown with two neighboring teeth, 504 and 506. The motion of neighboring tooth 504, including the corresponding bracket, is shown by dashed line 520. Dashed line 520 represents the movement of a centroid of neighboring tooth 504. Dashed line 522 represents the movement of a centroid of neighboring tooth 506. In some embodiments, the dashed lines 520 and 522 are determined based on an orthodontic treatment plan. In some embodiments, the dashed lines 520 and 522 represent the path of the tooth between a first treatment configuration and a second treatment configuration. For example, the first treatment configuration may be an initial treatment configuration, including tooth positions prior to the start of the orthodontic treatment or at a first stage of the orthodontic treatment. The second treatment configuration may be a final configuration, including the intended final positions for the teeth at the end of treatment or a later stage of the orthodontic treatment. As another example, the first treatment configuration and/or second treatment configuration may be an additional treatment configuration. The additional treatment configuration being a treatment configuration between an initial treatment configuration and a second treatment configuration. The additional treatment configuration may be determined from the orthodontic treatment plan.

FIG. 5C illustrates an example exclusion volume configuration 500, in accordance with some embodiments of the technology described herein. Exclusion volume 500 includes the exclusion volume 524 for the first neighboring tooth and exclusion volume 526 for the second neighboring tooth. Exclusion volume 524 is based on the movement of neighboring tooth 504 between the first and second treatment configuration, as represented by dashed line 520. Exclusion volume 526 is based on the movement of neighboring tooth 506 between the first and second treatment configuration, as represented by dashed line 522. In some embodiments, the exclusion volumes are based on the volume occupied by the tooth as it moved along the path 520 and 522, respectively. In some embodiments, the exclusion volume is based on merging the positions of teeth, as described herein.

In some embodiments, exclusion volume includes gingiva surface 528. Gingiva surface 528 is based on patient's gingiva 516. Gingiva surface 528 may be generated by applying a translation (e.g., a vertical offset) to patient's gingiva 516 to represent a degree of swelling of the patient's gingiva. In some embodiments, gingiva surface 528 may be generated by applying a scaling operation to patient's gingiva 516. In some embodiments, gingiva surface 528 may be generated by applying a thickening operation to patient's gingiva 516.

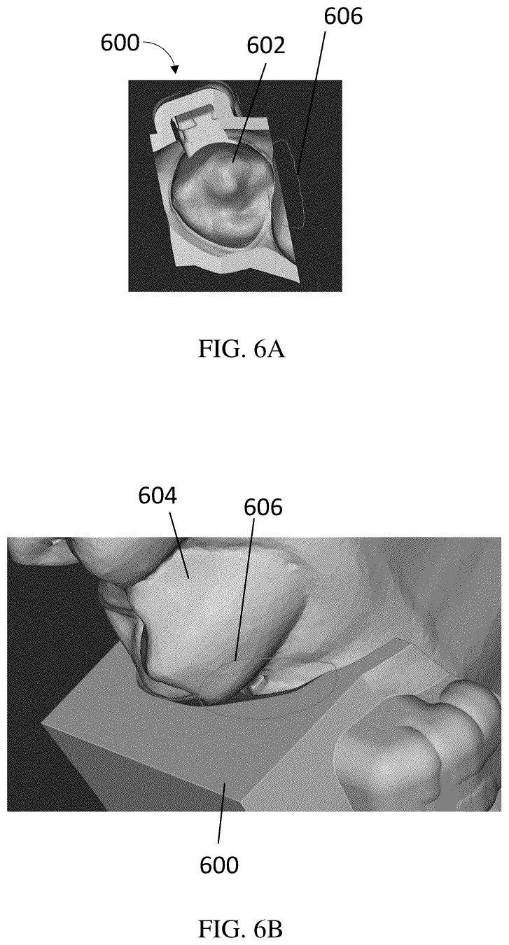

FIG. 6A illustrates an example of rebonding tray 600, in accordance with some embodiments of the technology described herein. Rebonding tray 600 includes internal cavity 602 for receiving the target tooth and recess 606 indicated by the circled region for providing clearance with a neighboring tooth of the target tooth.

FIG. 6B illustrates the example rebonding tray 600 of FIG. 6A placed over a target tooth. As shown in FIG. 6B, recess 606 illustrated by the circled region of rebonding tray 600 accommodates a side portion of neighboring tooth 604 such that the rebonding tray fits over the target tray without being obscured by neighboring tooth 604.

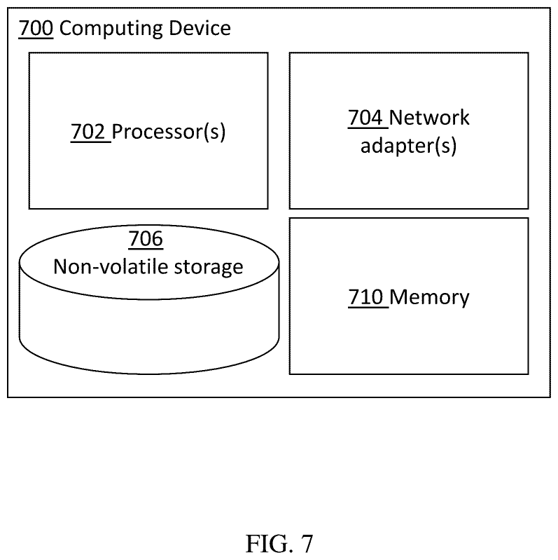

FIG. 7 illustrates an example implementation of a computer system 700 that may be used in connection with any of the embodiments of the disclosure provided herein. For example, process 300 may be performed using computing system 700. The computing system 700 may include one or more computer hardware processors 702 and one or more non-transitory computer-readable storage media. For example, one or more volatile storage devices 710 and/or one or more non-volatile storage devices 706 (e.g., a hard disk, a flash memory, etc.) may be included with computing system 700. The hardware processor 702 may control writing data to and reading data from the volatile storage device 710 and the non-volatile storage device 706, which may serve as non-transitory computer-readable storage media storing processor-executable instructions for execution by the hardware processor 702.

Computing system 700 may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone, tablet, or any other suitable portable or fixed electronic device, as aspects of the technology described herein are not limited in this respect.

Also, a computer may have one or more input and output devices. These devices may be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks, fiber optic networks, or any suitable combination thereof.

Having thus described several aspects of at least one embodiment of the technology described herein, it is to be appreciated that various alterations, modifications, and improvements will readily occur to those skilled in the art.

Such alterations, modifications, and improvements are intended to be part of this disclosure and are intended to be within the spirit and scope of disclosure. Further, though advantages of the technology described herein are indicated, not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

The above-described embodiments of the technology described herein may be implemented in any of numerous ways. For example, the embodiments may be implemented using hardware, software, or a combination thereof. When implemented in software, the software code may be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. However, a processor may be implemented using circuitry in any suitable format.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, aspects of the technology described herein may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments described above. As is apparent from the foregoing examples, a computer readable storage medium may retain information for a sufficient time to provide computer-executable instructions in a non-transitory form. Such a computer readable storage medium or media may be transportable, such that the program or programs stored thereon may be loaded onto one or more different computers or other processors to implement various aspects of the technology as described above. As used herein, the term “computer-readable storage medium” encompasses only a non-transitory computer readable medium that may be considered to be a manufacture (i.e., article of manufacture) or a machine.

The terms “program” or “software” are used herein in a generic sense to refer to any type of computer code or set of processor-executable instructions that may be employed to program a computer or other processor to implement various aspects of the technology as described above. Additionally, one or more computer programs that when executed perform methods of the technology described herein need not reside on a single computer or processor, but may be distributed in a modular fashion amongst a number of different computers or processors to implement various aspects of the technology described herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, for example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term). The phraseology and terminology used herein is for the purpose of description and should not be regarded as limiting. The use of “including,” “comprising,” “having,” “containing,” “involving,” and variations thereof, is meant to encompass the items listed thereafter and additional items.

Unless otherwise specified, the terms “approximately,” “substantially,” and “about” may be used to mean within ±10% of a target value in some embodiments. The terms “approximately,” “substantially” and “about” may include the target value.

Various aspects are described in this disclosure, which include but are not limited to the following aspects:

1. A method of designing a rebonding tray, the method comprising: using at least one computer hardware processor to perform: obtaining a first treatment configuration comprising a first position of a target tooth and a first position of a neighboring tooth of the target tooth; obtaining a second treatment configuration comprising a second position of the target tooth and a second position of the neighboring tooth of the target tooth; determining a mapping between the first treatment configuration and the second treatment configuration, wherein the mapping comprises a path of tooth movement for the neighboring tooth of the target tooth and corrects for movement of the target tooth; determining an exclusion zone based on the mapping between the first treatment configuration and the second treatment configuration; and determining a shape of the rebonding tray based on a shape of the target tooth and the exclusion zone.

2. The method of aspect 1, further comprising: obtaining one or more additional treatment configurations between the first treatment configuration and the second treatment configuration, wherein each of the one or more additional treatment configurations comprise respective positions of the target tooth and a respective position of the neighboring tooth of the target teeth; and determining the exclusion zone comprises mapping between the first treatment configuration, the one or more additional treatment configurations, and the second treatment configuration.

3. The method of any of aspects 1-2, wherein the first treatment configuration is determined based on a scan of the teeth in a patient's mouth.

4. The method of any of aspects 1-3, wherein the neighboring tooth of the target tooth comprises one of the patient's other teeth located in the patient's jaw with the target tooth.

5. The method of any of aspects 1-4, wherein the second treatment configuration comprises the target tooth positions for a patient's teeth at the end of an orthodontic treatment plan.

6. The method of any of aspects 1-5, wherein the mapping between the first treatment configuration and the second treatment configuration comprises determining a transformation between the second treatment configuration and the first treatment configuration such that the second position of the target tooth, in the second treatment configuration, is aligned with the first position of the target tooth, in the first treatment configuration.

7. The method of aspect 6, wherein the transformation between the second treatment configuration and the first treatment configuration is determined such that a bracket position of the target tooth in the second position is aligned with a bracket position in the first position.

8. The method of aspect 6, wherein the transformation between the second treatment configuration and the first treatment configuration is determined such that the centroid position of the target tooth in the second position is aligned with the centroid of the target tooth in the first position.

9. The method of any of aspects 1-8, wherein determining the exclusion zone comprises combining a volume of the neighboring tooth of the target tooth with a gingiva volume.

10. The method of aspect 9, wherein the gingiva volume is determined based on the scan of the teeth in a patient's mouth and a transformation.

11. The method of any of aspects 1-10, further comprising manufacturing a rebonding tray based on the determined shape of the rebonding tray, wherein manufacturing the rebonding tray comprises using additive manufacturing to manufacture the rebonding tray.

12. The method of any of aspects 1-11, wherein the exclusion zone includes a first position and a second position of hardware mounted to the target tooth and/or the neighboring tooth of the target tooth in the first treatment configuration and the second treatment configuration.

13. The method of any of aspects 12, wherein determining the exclusion zone based on the mapping between the first treatment configuration and the second treatment configuration further comprises using one or more additional teeth located in the patient's jaw with the target tooth.

14. A system comprising at least one hardware processor; and at least one non-transitory computer-readable storage medium storing processor executable instructions that, when executed by the at least one computer hardware processor, cause the at least one computer hardware processor to perform a method for manufacturing a rebonding tray, the method comprising: obtaining a first treatment configuration comprising a first position of a target tooth and first position of a neighboring tooth of the target tooth; obtaining a second treatment configuration comprising a second position of the target tooth and a second position of the neighboring tooth of the target tooth; determining a mapping between the first treatment configuration and the second treatment configuration, wherein the mapping comprises the path of tooth movement for the neighboring tooth of the target tooth; determining an exclusion zone based on the mapping between the first treatment configuration and the second treatment configuration; and determining a shape of a rebonding tray based on a shape of the target tooth and the exclusion zone.

15. The system of aspect 14, wherein the method further comprises: obtaining one or more additional treatment configurations between the first treatment configuration and the second treatment configuration, wherein each of the one or more additional treatment configurations comprise respective positions of the target tooth and a respective position of the neighboring tooth of the target teeth; and determining the exclusion zone comprises mapping between the first treatment configuration, the one or more additional treatment configurations, and the second treatment configuration.

16. The system of any of aspects 14-15, wherein the first treatment configuration is determined based on a scan of the teeth in a patient's mouth.

17. The system of any of aspects 14-16, wherein the neighboring tooth of the target tooth comprises one of the patient's other teeth located in the patient's jaw with the target tooth.

18. The system of any of aspects 14-17, wherein the second treatment configuration comprises the target tooth positions for a patient's teeth at the end of an orthodontic treatment plan.

19. The system of any of aspects 14-18, wherein the mapping between the first treatment configuration and the second treatment configuration comprises determining a transformation between the second treatment configuration and the first treatment configuration such that the second position of the target tooth, in the second treatment configuration, is aligned with the first position of the target tooth, in the first treatment configuration.

20. The system of aspect 19, wherein the transformation between the second treatment configuration and the first treatment configuration is determined such that a bracket position of the target tooth in the second position is aligned with a bracket position in the first position.

21. The method of aspect 19, wherein the transformation between the second treatment configuration and the first treatment configuration is determined such that the centroid position of the target tooth in the second position is aligned with the centroid of the target tooth in the first position.

22. The system of any of aspects 14-21, wherein determining the exclusion zone comprises combining a volume of the neighboring tooth of the target tooth with a gingiva volume.

23. The system of aspect 22, wherein the gingiva volume is determined based on the scan of the teeth in a patient's mouth and a transformation.

24. The system of any of aspects 14-23, wherein the method further comprises manufacturing a rebonding tray based on the determined shape of the rebonding tray, wherein manufacturing the rebonding tray comprises using additive manufacturing to manufacture the rebonding tray.

25. The method of any of aspects 14-24, wherein the exclusion zone includes a first position and a second position of hardware mounted to the target tooth and/or the neighboring tooth of the target tooth in the first treatment configuration and the second treatment configuration.

26. The method of any of aspects 14-25, wherein determining the exclusion zone based on the mapping between the first treatment configuration and the second treatment configuration further comprises using one or more additional teeth located in the patient's jaw with the target tooth.

27. At least one non-transitory computer-readable storage medium storing processor executable instructions that, when executed by at least one computer hardware processor, cause the at least one computer hardware processor to perform a method for manufacturing a rebonding tray, the method comprising: obtaining a first treatment configuration comprising a first position of a target tooth and first position of a neighboring tooth of the target tooth; obtaining a second treatment configuration comprising a second position of the target tooth and a second position of the neighboring tooth of the target tooth; determining a mapping between the first treatment configuration and the second treatment configuration, wherein the mapping comprises the path of tooth movement for the neighboring tooth of the target tooth; determining an exclusion zone based on the mapping between the first treatment configuration and the second treatment configuration; and determining a shape of a rebonding tray based on a shape of the target tooth and the exclusion zone.

28. The at least one non-transitory storage medium of aspect 27, wherein the method further comprises: obtaining one or more additional treatment configurations between the first treatment configuration and the second treatment configuration, wherein each of the one or more additional treatment configurations comprise respective positions of the target tooth and a respective position of the neighboring tooth of the target teeth; and determining the exclusion zone comprises mapping between the first treatment configuration, the one or more additional treatment configurations, and the second treatment configuration.

29. The at least one non-transitory storage medium of any of aspects 27-28, wherein the first treatment configuration is determined based on a scan of the teeth in a patient's mouth.

30. The at least one non-transitory storage medium of any of aspects 27-29, wherein the neighboring tooth of the target tooth comprises one of the patient's other teeth located in the patient's jaw with the target tooth.

31. The at least one non-transitory storage medium of any of aspects 27-30, wherein the second treatment configuration comprises the target tooth positions for a patient's teeth at the end of an orthodontic treatment plan.

32. The at least one non-transitory storage medium of any of aspects 27-31, wherein the mapping between the first treatment configuration and the second treatment configuration comprises determining a transformation between the second treatment configuration and the first treatment configuration such that the second position of the target tooth, in the second treatment configuration, is aligned with the first position of the target tooth, in the first treatment configuration.

33. The at least one non-transitory storage medium of aspect 32, wherein the transformation between the second treatment configuration and the first treatment configuration is determined such that a bracket position of the target tooth in the second position is aligned with a bracket position in the first position.

34. The at least one non-transitory storage medium of aspect 32, wherein the transformation between the second treatment configuration and the first treatment configuration is determined such that the centroid position of the target tooth in the second position is aligned with the centroid of the target tooth in the first position.

35. The at least one non-transitory storage medium of any of aspects 27-34, wherein determining the exclusion zone comprises combining a volume of the neighboring tooth of the target tooth with a gingiva volume.

36. The at least one non-transitory storage medium of aspect 35, wherein the gingiva volume is determined based on the scan of the teeth in a patient's mouth and a transformation.

37. The at least one non-transitory storage medium of any of aspects 27-36, wherein the method further comprises manufacturing a rebonding tray based on the determined shape of the rebonding tray, wherein manufacturing the rebonding tray comprises using additive manufacturing to manufacture the rebonding tray.

38. The at least one non-transitory storage medium of any of aspects 27-37, wherein the exclusion zone includes a first position and a second position of hardware mounted to the target tooth and/or the neighboring tooth of the target tooth in the first treatment configuration and the second treatment configuration.

39. The at least one non-transitory storage medium of any of aspects 27-38, wherein determining the exclusion zone based on the mapping between the first treatment configuration and the second treatment configuration further comprises using one or more additional teeth located in the patient's jaw with the target tooth.

40. A rebonding tray, the rebonding tray being manufactured according to a method of manufacturing a rebonding tray, the method comprising: obtaining a first treatment configuration comprising a first position of a target tooth and first positions of a neighboring tooth of the target tooth; obtaining a second treatment configuration comprising a second position of the target tooth and second the neighboring tooth of the target tooth; determining a mapping between the first treatment configuration and the second treatment configuration, wherein the mapping comprises a path of tooth movement for the neighboring tooth of the target tooth and corrects for movement of the target tooth; determining an exclusion zone based on the mapping between the first treatment configuration and the second treatment configuration; determining a shape of a rebonding tray based on a shape of the target tooth and the exclusion zone; and manufacturing a rebonding tray based on the determined shape of the rebonding tray.

41. The rebonding tray of aspect 40, wherein the method further comprises: obtaining one or more additional treatment configurations between the first treatment configuration and the second treatment configuration, wherein each of the one or more additional treatment configurations comprise respective positions of the target tooth and a respective position of the neighboring tooth of the target teeth; and determining the exclusion zone comprises mapping between the first treatment configuration, the one or more additional treatment configurations, and the second treatment configuration.

42. The rebonding tray of any of aspects 40-41, wherein the first treatment configuration is determined based on a scan of the teeth in a patient's mouth.

43. The rebonding tray of any of aspects 40-42, wherein the neighboring tooth of the target tooth comprises one of the patient's other teeth located in the patient's jaw with the target tooth.

44. The rebonding tray of any of aspects 40-43, wherein the second treatment configuration comprises the target tooth positions for a patient's teeth at the end of an orthodontic treatment plan.

45. The rebonding tray of any of aspects 40-44, wherein the mapping between the first treatment configuration and the second treatment configuration comprises determining a transformation between the second treatment configuration and the first treatment configuration such that the second position of the target tooth, in the second treatment configuration, is aligned with the first position of the target tooth, in the first treatment configuration.

46. The rebonding tray of aspect 45, wherein the transformation between the second treatment configuration and the first treatment configuration is determined such that a bracket position of the target tooth in the second position is aligned with a bracket position in the first position.

47. The rebonding tray of aspect 45, wherein the transformation between the second treatment configuration and the first treatment configuration is determined such that the centroid position of the target tooth in the second position is aligned with the centroid of the target tooth in the first position.

48. The rebonding tray of any of aspects 40-47, wherein determining the exclusion zone comprises combining a volume of the neighboring tooth of the target tooth with a gingiva volume.

49. The rebonding tray of aspect 48, wherein the gingiva volume is determined based on the scan of the teeth in a patient's mouth and a transformation.

50. The rebonding tray of any of aspects 40-49, wherein the method further comprises manufacturing a rebonding tray based on the determined shape of the rebonding tray, wherein manufacturing the rebonding tray comprises using additive manufacturing to manufacture the rebonding tray.

51. The rebonding tray of any of aspects 40-50, wherein the exclusion zone includes a first position and a second position of hardware mounted to the target tooth and/or the neighboring tooth of the target tooth in the first treatment configuration and the second treatment configuration.

52. The rebonding tray of any of aspects 40-51, wherein determining the exclusion zone based on the mapping between the first treatment configuration and the second treatment configuration further comprises using one or more additional teeth located in the patient's jaw with the target tooth.

53. The rebonding tray of any of aspects 40-52, wherein the exclusion zone is a volume based on a volume occupied by one or more teeth along the mapping between the first treatment configuration and the second treatment configuration.

54. The rebonding tray of any of aspects 40-53, wherein the exclusion zone is one or more planes that are based on the mapping between the first treatment configuration and the second treatment configuration.

55. The rebonding tray of any of aspects 40-54, wherein the exclusion zone is a three-dimensional surface based on the mapping between the first treatment configuration and the second treatment configuration.

1. A system comprising at least one hardware processor; and at least one non-transitory computer-readable storage medium storing processor executable instructions that, when executed by the at least one computer hardware processor, cause the at least one computer hardware processor to perform a method for manufacturing a rebonding tray, the method comprising:
obtaining a first treatment configuration comprising a first position of a target tooth and first position of a neighboring tooth of the target tooth;
obtaining a second treatment configuration comprising a second position of the target tooth and a second position of the neighboring tooth of the target tooth;
determining a mapping between the first treatment configuration and the second treatment configuration, wherein the mapping comprises the path of tooth movement for the neighboring tooth of the target tooth;
determining an exclusion zone based on the mapping between the first treatment configuration and the second treatment configuration; and
determining a shape of a rebonding tray based on a shape of the target tooth and the exclusion zone.

2. The system of claim 1, wherein the method further comprises:
obtaining one or more additional treatment configurations between the first treatment configuration and the second treatment configuration, wherein each of the one or more additional treatment configurations comprise respective positions of the target tooth and a respective position of the neighboring tooth of the target teeth; and
determining the exclusion zone comprises mapping between the first treatment configuration, the one or more additional treatment configurations, and the second treatment configuration.

3. The system of claim 1, wherein the first treatment configuration is determined based on a scan of the teeth in a patient's mouth.

4. The system of claim 1, wherein the neighboring tooth of the target tooth comprises one of the patient's other teeth located in the patient's jaw with the target tooth.

5. The system of claim 1, wherein the second treatment configuration comprises the target tooth positions for a patient's teeth at the end of an orthodontic treatment plan.

6. The system of claim 1, wherein the mapping between the first treatment configuration and the second treatment configuration comprises determining a transformation between the second treatment configuration and the first treatment configuration such that the second position of the target tooth, in the second treatment configuration, is aligned with the first position of the target tooth, in the first treatment configuration.

7. The system of claim 1, wherein determining the exclusion zone comprises combining a volume of the neighboring tooth of the target tooth with a gingiva volume.

8. The method of claim 1, wherein the exclusion zone includes a first position and a second position of hardware mounted to the target tooth and/or the neighboring tooth of the target tooth in the first treatment configuration and the second treatment configuration.

9. The method of claim 1, wherein determining the exclusion zone based on the mapping between the first treatment configuration and the second treatment configuration further comprises using one or more additional teeth located in the patient's jaw with the target tooth.

10. At least one non-transitory computer-readable storage medium storing processor executable instructions that, when executed by at least one computer hardware processor, cause the at least one computer hardware processor to perform a method for manufacturing a rebonding tray, the method comprising:
obtaining a first treatment configuration comprising a first position of a target tooth and first position of a neighboring tooth of the target tooth;
obtaining a second treatment configuration comprising a second position of the target tooth and a second position of the neighboring tooth of the target tooth;
determining a mapping between the first treatment configuration and the second treatment configuration, wherein the mapping comprises the path of tooth movement for the neighboring tooth of the target tooth;
determining an exclusion zone based on the mapping between the first treatment configuration and the second treatment configuration; and
determining a shape of a rebonding tray based on a shape of the target tooth and the exclusion zone.

11. A rebonding tray, the rebonding tray being manufactured according to a method of manufacturing a rebonding tray, the method comprising:
obtaining a first treatment configuration comprising a first position of a target tooth and first positions of a neighboring tooth of the target tooth;
obtaining a second treatment configuration comprising a second position of the target tooth and second the neighboring tooth of the target tooth;
determining a mapping between the first treatment configuration and the second treatment configuration, wherein the mapping comprises a path of tooth movement for the neighboring tooth of the target tooth and corrects for movement of the target tooth;
determining an exclusion zone based on the mapping between the first treatment configuration and the second treatment configuration;
determining a shape of a rebonding tray based on a shape of the target tooth and the exclusion zone; and
manufacturing a rebonding tray based on the determined shape of the rebonding tray.

12. The rebonding tray of claim 11, wherein the method further comprises:
obtaining one or more additional treatment configurations between the first treatment configuration and the second treatment configuration, wherein each of the one or more additional treatment configurations comprise respective positions of the target tooth and a respective position of the neighboring tooth of the target teeth; and
determining the exclusion zone comprises mapping between the first treatment configuration, the one or more additional treatment configurations, and the second treatment configuration.

13. The rebonding tray of claim 11, wherein the first treatment configuration is determined based on a scan of the teeth in a patient's mouth.

14. The rebonding tray of claim 11, wherein the second treatment configuration comprises the target tooth positions for a patient's teeth at the end of an orthodontic treatment plan.

15. The rebonding tray of claim 11, wherein the mapping between the first treatment configuration and the second treatment configuration comprises determining a transformation between the second treatment configuration and the first treatment configuration such that the second position of the target tooth, in the second treatment configuration, is aligned with the first position of the target tooth, in the first treatment configuration.

16. The rebonding tray of claim 15, wherein the transformation between the second treatment configuration and the first treatment configuration is determined such that a bracket position of the target tooth in the second position is aligned with a bracket position in the first position.

17. The rebonding tray of claim 15, wherein the transformation between the second treatment configuration and the first treatment configuration is determined such that the centroid position of the target tooth in the second position is aligned with the centroid of the target tooth in the first position.

18. The rebonding tray of claim 11, wherein the exclusion zone is a volume based on a volume occupied by one or more teeth along the mapping between the first treatment configuration and the second treatment configuration.

19. The rebonding tray of claim 11, wherein the exclusion zone is one or more planes that are based on the mapping between the first treatment configuration and the second treatment configuration.

20. The rebonding tray of claim 11, wherein the exclusion zone is a three-dimensional surface based on the mapping between the first treatment configuration and the second treatment configuration.
